(19) United States
(12) Patent Application Publication
(10) Pub. No.: US 2007/0233259 A1
(43) Pub. Date: Oct. 4, 2007

(54) INTERVERTEBRAL DISC PROSTHESIS AND METHODS OF IMPLANTATION

(76) Inventors: Nabil L. Muhanna, Gainesville, GA (US); David L. Schalliol, Oakwood, GA (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(21) Appl. No.: 11/754,611

(22) Filed: May 29, 2007

Related U.S. Application Data
(63) Continuation of application No. 11/248,395, filed on Oct. 12, 2005, now abandoned.
Application No. 11/248,395 is a continuation-in-part of application No. 11/176,614, filed on Jul. 7, 2005, now abandoned.
Application No. 11/176,614 is a continuation of application No. 10/047,587, filed on Jan. 15, 2002, now Pat. No. 6,936,070.
(60) Provisional application No. 60/262,974, filed on Jan. 17, 2001.
(60) Provisional application No. 60/723,646, filed on Oct. 5, 2005.

(51) Int. Cl. A61F 2/44

(52) U.S. Cl. 623/17.12; 623/17.16

(57) ABSTRACT

Various materials, constructs, and methods for maintaining an intervertebral space are provided.

### CROSS REFERENCE TO RELATED APPLICATIONS

This application is a continuation of U.S. patent application Ser. No. 11/248,395, filed on Oct. 12, 2005, which application is a continuation-in-part of U.S. patent application Ser. No. 11/176,614, filed on Jul. 7, 2005, which is a continuation of U.S. patent application Ser. No. 10/047,587, filed on Jan. 15, 2002, now U.S. Pat. No. 6,937,070; which application claims the benefit of U.S. Provisional Application No. 60/262,974, filed Jan. 17, 2001, each of which is incorporated herein by this reference in its entirety; and U.S. patent application Ser. No. 11/248,395, filed on Oct. 12, 2005 claims the benefit of U.S. Provisional Application No. 60/723,646, filed on Oct. 5, 2005; which is incorporated herein in its entirety by this reference.

### TECHNICAL FIELD

The present invention generally relates to various prostheses for at least a portion of a damaged intervertebral disc and methods for implanting the intervertebral disc prostheses into the spinal column.

### BACKGROUND

The spinal column, which is the central support to the vertebrate skeleton and a protective enclosure for the spinal cord, is a linear series of bones, or vertebrae. Intervertebral discs separate and reduce friction between adjacent vertebrae and absorb compression forces applied to the spinal column. Spinal nerves that extend from each side of the spinal cord exit the column at intervertebral forama.

A typical vertebra comprises an anterior body, and a posterior arch that surrounds the spinal cord lying within the vertebral foramen formed by the arch. The muscles that flex the spine are attached to three processes extending from the posterior arch. On the upper surface of each vertebra in a standing human are two superior articulated processes that oppose two inferior articulated processes extending from the lower surface of an adjacent vertebra. Facets on the opposing processes determine the range and direction of movement between adjacent vertebrae, and hence the flexibility of the spinal column.

The intervertebral discs include the fibrillar cartilage of the anulus fibrosus, a fibrous ring, the center of which is filled with an elastic fibrogelatinous pulp that acts as a shock absorber. The outer third of the anulus fibrosus is innervated. The entire spinal column is united and strengthened by encapsulating ligaments.

Back pain is one of the most significant problems facing the workforce in the United States today. It is a leading cause of sickness-related absenteeism and is the main cause of disability for people between ages 19 and 45. Published reports suggest that the economic cost is significant, treatment alone exceeding $80 billion annually. Although acute back pain is common and typically treated with analgesics, chronic pain may demand surgery for effective treatment.

Back pain can occur from pinching or irritation of spinal nerves, compression of the spine, vertebral shifting relative to the spinal cord axis, and bone spur formation. The most common cause of disabling back pain, however, stems from trauma to an intervertebral disc, resulting from mechanical shock, stress, tumors, or degenerative disease, which may impair functioning of the disc and limit spinal mobility. In many cases, the disc is permanently damaged and the preferred treatment becomes partial or total excision.

Another cause of back injury is herniation of the intervertebral disc, wherein the gelatinous fluid of the nucleus pulposus enters the vertebral canal and pressures the spinal cord. Again, surgery is often the only method available for permanent relief from pain or the neurological damage ensuing from the pressure of fluid on the spinal cord, and requires replacement of the damaged disc.

Traumatic injury to an intervertebral disc that is not removed will frequently promote scar tissue formation. Scar tissue is weaker than original healthy tissue, so the disc will progressively degenerate, lose water content, stiffen and become less effective as a shock absorber. Eventually, the disc may deform, herniate, or collapse, limiting flexibility of the spinal column at that position. The only option is for the intervertebral disc to be partially or totally removed.

When the disc is partially or completely removed, it is necessary to replace the excised material to prevent direct contact between hard bony surfaces of adjacent vertebrae. One vertebral spacer that may be inserted between adjacent vertebrae, according to U.S. Pat. No. 5,989,291 to Ralph et al., includes two opposing plates separated by a belleville washer or a modified belleville washer. The washer provides a restorative force to mimic the natural function of the disc by providing a shock absorber and mobility between adjacent vertebrae. An alternative approach is a “cage” that maintains the space usually occupied by the disc to prevent the vertebrae from collapsing and impinging the nerve roots. However, mechanical devices intended to replicate intervertebral disc function have had only limited success.

Spinal fusion may be used to restrict the motion between two vertebrae that comes from segmental instability. Fusing the vertebrae together reduces the mechanical back pain by preventing the now immobile vertebrae from impinging on the spinal nerve. The disadvantage of such spacers is that stability is created at the expense of the flexibility of the spine.

Surgical procedures for replacing intervertebral disc material, rather than fusing of the vertebrae, have included both anterior approaches and posterior approaches to the spinal column. The posterior approach (from the back of the patient) encounters the spinous process, superior articular process, and the inferior articular process that must be removed to allow insertion of the disc replacement material into the intervertebral space. The anterior approach to the spinal column is complicated by the internal organs that must be bypassed or circumvented to access the vertebrae.

Many intervertebral spacers require preparation of the surfaces of the adjacent vertebrae to accommodate the spacer, causing significant tissue and bone trauma. For example, chiseling or drilling of the vertebral surface may be required to prepare a receiving slot. They may also require screwing the spacer into the intervertebral space, making installation difficult and increasing trauma to the vertebral tissue. Many spacers include complex geometries and are costly to manufacture. Examples of such geometrically complex spacers are described in U.S. Pat. No. 5,609,636 to Kohrs et al., U.S. Pat. No. 5,780,919 to Zdeblick et al., U.S. Pat. No. 5,865,848 to Baker, and U.S. Pat. No. 5,776,196 to Matsuzaki et al.

### SUMMARY

The present invention is directed generally to various materials, constructs, and methods used to alleviate numerous vertebral conditions and injuries. Depending on the needs of the particular patient, the present invention contemplates complete, substantial, or partial replacement of the intervertebral disc. According to some aspects of the invention, an intervertebral disc or intervertebral spacer provides cushioning and support between vertebrae. According to some other aspects of the invention, an injectable substance is used to fill at least partially the interior of an intervertebral disc. In such aspects, little or no disc needs to be removed prior to injection of the filler material. Instead, an incision is made in the disc to receiving a suitable filler material therethrough.

The various intervertebral disc prostheses of the present invention may be used to replace all or a portion of an intervertebral disc that has degenerated due to traumatic injury, vertebral displacement, disease such as, for example, autoimmune disease or rheumatoid arthritis, or any other abnormal condition of the spinal column that may injure or shift the intervertebral disc, and to provide support to the vertebrae. Depending on degree of damage to the intervertebral disc, the location of the damage, and needs of the patient, the shape, size, type, and configuration of the prosthesis used may be selected to obtain the desired degree of flexibility, compressibility, and resilience needed to provide sufficient shock absorbance protection to the spinal cord.

The various prostheses according to the present invention can be inserted relatively easily by the surgeon into the intervertebral space while minimizing trauma to the opposing surfaces of the vertebrae and to the bony processes.

In one aspect, an intervertebral disc prosthesis comprises a body adapted to fit within an intervertebral space between adjacent vertebrae, wherein the body comprises a resilient biocompatible material. The resilient biocompatible material may be a dissected human or animal tissue, an inorganic polymer, an organic polymer, or any combination thereof.

In another aspect, the present invention provides various biocompatible intervertebral disc prostheses that are resilient to compressive forces that may be adapted to fit within an intervertebral space and, when implanted in the spinal column of a patient, will maintain the separation between adjacent vertebrae, provide shock absorbent protection, and allow flexibility of the spinal column.

The present invention further provides methods for the implantation of the intervertebral disc prosthesis of the present invention into the spinal column of a human or animal patient.

In one aspect, a method of maintaining an intervertebral space between adjacent vertebrae comprises excising at least a portion of an intervertebral disc, thereby creating a receiving slot, and inserting into the receiving slot at least one intervertebral disc prosthesis comprising a resilient biocompatible material according to the present invention. If needed, a minimal portion of the bony process of a vertebra may be removed to create access to the damaged intervertebral disc.

The present invention is also directed to an interior disc prosthesis. In one aspect, an interior disc prosthesis comprises a biocompatible, injectable material inserted into the interior of an intervertebral disc through an incision in an exterior of the intervertebral disc, wherein a minimal portion of the exterior of the disc is removed prior to insertion of the biocompatible, injectable material.

Further, the present invention encompasses a filler material for an intervertebral disc. The filler material comprises a biocompatible material capable of being injected into an interior of an intervertebral disc through an incision in an exterior of the intervertebral disc.

In yet another aspect, a method of maintaining an intervertebral space between adjacent vertebrae comprises making an incision in an intervertebral disc, the disc having an exterior and an interior, and injecting through the incision a biocompatible filler material.

Various other aspects, objects, features, and advantages of the invention will become more apparent upon review of the detailed description set forth below when taken in conjunction with the accompanying drawing figures, which are briefly described as follows.

### BRIEF DESCRIPTION OF THE DRAWINGS

The description refers to the accompanying drawings in which like reference characters refer to like parts throughout the several views, and in which:

FIG. 1 is an exploded perspective view of a portion of the human spinal column, showing the body of an intervertebral disc prosthesis according to the present invention inserted between adjacent vertebrae;

FIG. 2 is a side-elevation of the intervertebral disc prosthesis shown in FIG. 1, illustrating an intervertebral disc prosthesis inserted between two adjacent vertebrae, wherein an anterior face of the prosthesis is thicker than the posterior face;

FIG. 3A shows a perspective view illustrating an intervertebral disc prosthesis in the form of a folded sheet of detoxified resilient biocompatible material secured by a suture;

FIG. 3B shows a perspective view of a folded sheet intervertebral disc prosthesis between two adjacent vertebrae as viewed from the front (anterior) of the patient;

FIG. 3C shows a perspective side-view of a folded sheet intervertebral disc prosthesis between two adjacent vertebrae;

FIG. 4A illustrates the implantation of an intervertebral disc prosthesis, in the form of a ribbon of resilient biocompatible material, into an incision in an intervertebral disc; and

FIG. 4B illustrates a longitudinal section through two adjacent vertebrae and the intervertebral disc and showing, in situ, an intervertebral disc prosthesis, in the form of a ribbon of resilient biocompatible material confined within an intervertebral disc.

### DETAILED DESCRIPTION

A full and enabling disclosure of the present invention, including the best mode known to the inventor of carrying out the invention, is set forth more particularly in the remainder of the specification, including reference to the accompanying drawings, wherein like reference numerals designate corresponding parts throughout several figures. However, it should be understood that use of like numerals is not to be construed as an acknowledgment or admission that such features are necessarily identical or equivalent in any manner. This description is made for the purpose of illustrating the general principles of the invention and should not be taken as limiting in any manner. It also will be understood that where similar features are depicted, not all of such identical features may be labeled on the figures.

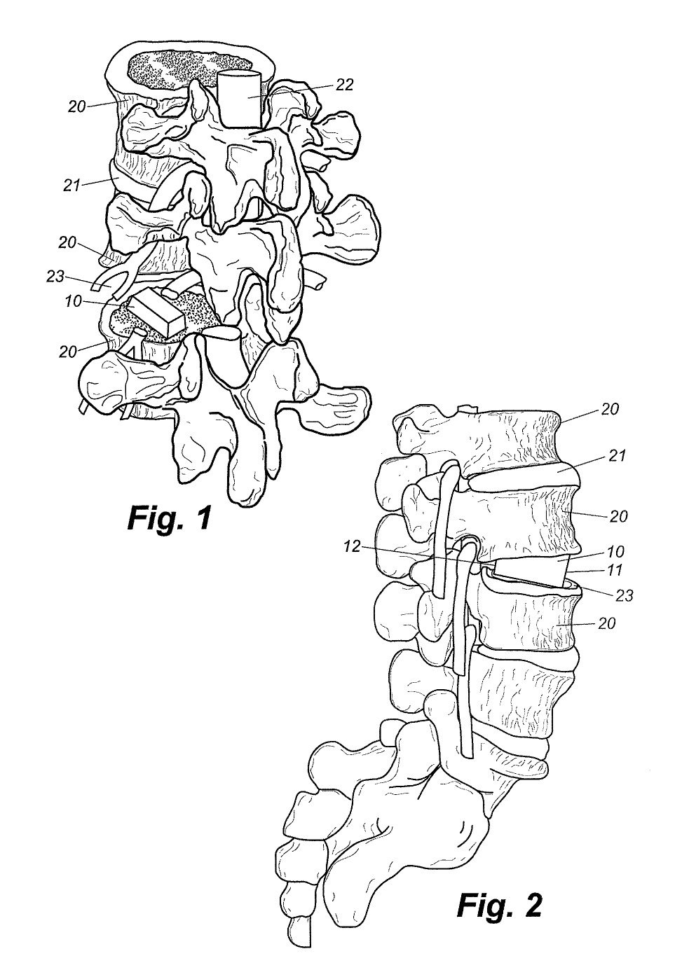

According to a first aspect of the present invention, a substantial or complete intervertebral disc replacement or prosthesis 10 may be used to support adjacent vertebrae 20 after partial or total surgical excision of an intervertebral disc 21. It is contemplated that the prosthesis 10 may be configured to be compressible and, therefore, capable of absorbing compressibility forces applied to the spinal column 22 of the patient. In the example illustrated in FIG. 1, the intervertebral disc prosthesis 10 includes a substantially rectangular body adapted to fit within the intervertebral space 23 between adjacent vertebrae 20. However, it will be understood that the prosthesis may have any geometrical form including, but not limited to, a square, circle, pillow, toroid, cube, or an annulus, provided that the form of the body is capable of maintaining the intervertebral space 23, while allowing mobility and flexibility of the spinal column.

In the example shown in FIG. 1, the intervertebral disc prosthesis 10 has multiple surfaces, including an anterior face 11 and a posterior face 12, wherein the anterior face 11 is directed towards the inner body cavity of the patient, and the posterior face 12 is directed towards the dorsal surface of the patient. The intervertebral disc prosthesis 10 may be configured such that the thickness of the anterior face 11 is greater than the thickness of the posterior face 12, as is illustrated in FIG. 2, or such that the thickness of the posterior face 12 is greater than the thickness of the anterior face 11 (not shown). The difference in the thickness of the opposing anterior 11 and posterior 12 faces of the intervertebral disc prosthesis 10 of the present invention may be selected to preserve the natural curvature of the spinal column.

In this and other aspects of the invention, the material of the intervertebral disc prosthesis of the present invention may be any biocompatible material having a degree of resilience that can provide a level of shock absorbance when the prosthesis is implanted in the spinal column of a patient. It will be understood that a biocompatible material is non-toxic to the tissues of a recipient human or animal patient, and will elicit a non-injurious immune reaction, or is non-immunogenic. A biocompatible material may support the invasion of cells from the recipient patient's own tissues into the intervertebral disc prosthesis. Such biocompatible materials may be selected from, but are not limited to, a tissue dissected from a human or animal, a synthetic organic or synthetic inorganic polymer, or any combination thereof. An example of a particularly useful biocompatible tissue is a human or animal pericardium. One example of a biocompatible tissue that may be particularly useful with the present invention is the treated bovine pericardium DURA SHIELD™ (Shelhigh, Inc., Milburn, N.J.). Examples of synthetic materials that may be useful in the present invention include GORLEX™ and PROLEAN™.

The dissected tissue that comprises the intervertebral disc prosthesis of the present invention may be treated to reduce immunogenicity, which is the ability of an implanted tissue to elicit an immune response in a recipient human or animal. Furthermore, the dissected tissue may be treated with a protein cross-linking agent such as, but not limited to, glutaraldehyde before implantation into the patient.

Any biocompatible tissue may be further treated with an anti-calcification process so that the tissue does not harden, stiffen, or otherwise become brittle after implantation into the intervertebral space or treated with an anti-coagulant such as, but not limited to, heparin.

Any treated biocompatible material for use in an intervertebral disc prosthesis of the present invention may be rendered non-toxic to the recipient patient by a detoxification process, especially, but not only, after treatment of a dissected tissue from a human or animal with a fixing agent. It also is contemplated that the intervertebral disc prostheses of the present invention can be sterilized, before implantation into a patient, by any suitable method known to those skilled in the art and that will not degrade any of the desired mechanical or biological properties of the prosthesis. An example of a method of sterilization and storage of an intervertebral disc prosthesis of the present invention is soaking in benzyl alcohol, which rapidly evaporates before insertion into the patient.

Examples of tissue detoxification and anti-calcification treatment processes, such as the NO-REACT® process (Shelhigh, Inc., Milburn, N.J.), and the design and construction of a detoxified biocompatible tissue such as DURA SHIELD™ (Shelhigh, Inc., Milburn, N.J.), that may be suitable materials for the intervertebral disc prosthesis of the present invention are described in Abolhoda et al., Ann. Thoracic Surg. 62, 1724-1730 (1996); Abolhoda et al., Ann. Thoracic Surg. 62, 169-174 (1996); Infantes & Angell. Adv. Anticalcific and Antidegenerative Treatment of Heart Valve Bioprostheses, pp. 221-231, ed: Gabbayid S. & Wheatley D. J, pub: Silent Partners, Inc., Austin (1997), each of which is incorporated herein by reference in its entirety.

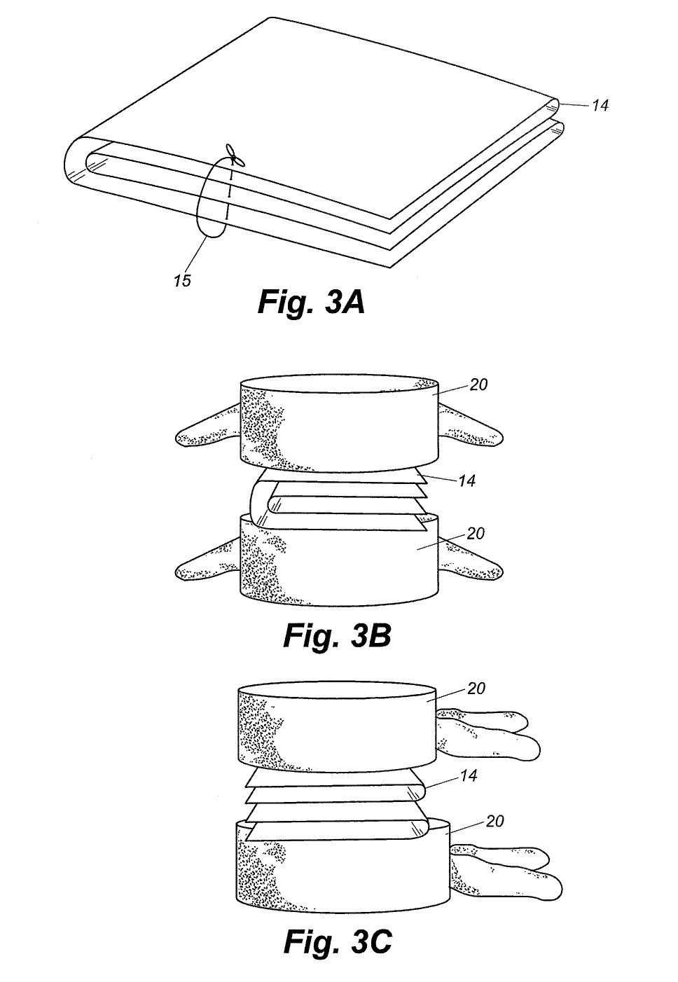

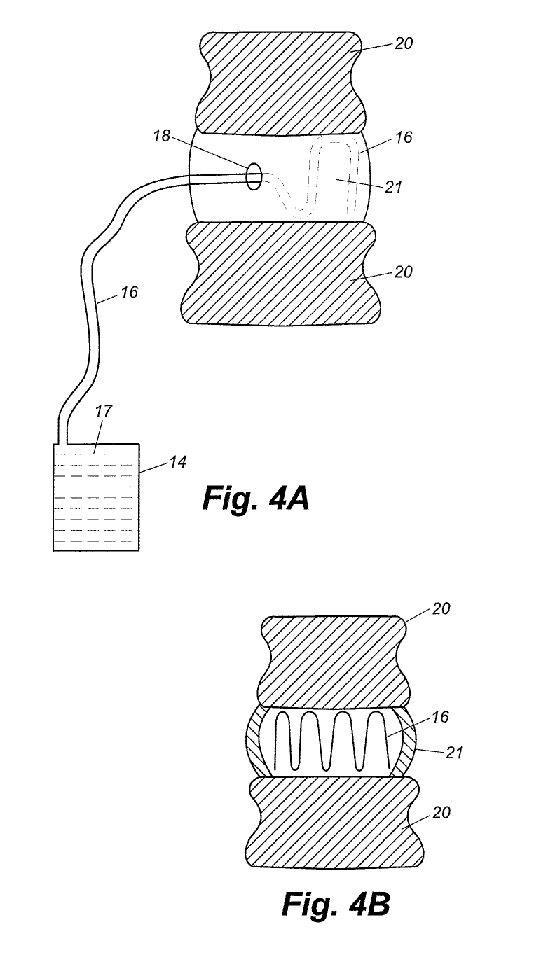

Turning to FIGS. 3A to 3C, in another example of this aspect of the present invention, the prosthesis comprises a folded sheet 14 of biocompatible material. The sheet 14 can be further adapted, folded, or otherwise manipulated to fit within and maintain an intervertebral space 23 between adjacent vertebrae 20, as shown in FIGS. 3B and 3C. The sheet 14 can be secured within the intervertebral space 23 by a first fastener 15 including, but not limited to, a staple, suture, an adhesive, or any other fastening material that can prevent further unfolding of the sheet 14, and that will not significantly impede movement of the vertebrae. Optionally, unfolding of the sheet 14 can be prevented by a first fastener 15 and the prosthesis 10 secured within the intervertebral space 23 by a second fastener.

In still another example, the intervertebral disc prosthesis may be a stack or laminate (not shown) comprising a plurality of layers of the biocompatible material. The plurality of the laminated layers can be secured by a first fastener such as, but not limited to, a staple, a suture, an adhesive, or by fusing the layers together to form the laminate body or cultivating cells within and between the layers, thereby holding the layers in the laminate.

In this and other aspects of the present invention, the intervertebral disc prosthesis may be supplemented using one or more augmenting substances that, in the intervertebral space, have a consistency ranging from a semi-solid state to a solid state. Any augmenting substance may be used in accordance with the present invention. Examples of substances that may be suitable include, but are not limited to, a silicone-based polymer, a methyl acrylate polymer, a collagen-based gel, a plastic, or any combination thereof. By using an augmenting substance, an intervertebral disc prosthesis may be modified as needed to provide the desired level of cushioning and shock absorbency. Thus, for example, a layered structure including sheets of a biocompatible material may be enlarged by delivering one or more of such augmenting substances between one or more layers, thereby making the prosthesis more full or plump. As another example, a prosthesis having the shape of a toroid may be configured to receive an augmenting substance within the central opening thereof. It will be understood that other prosthesis structures also may be augmented using one or more augmenting substances in any configuration, as desired.

It is contemplated by the present invention that the biocompatible material of the intervertebral disc prosthesis can be invaded by cells. The proliferating cells may be derived from the patient, or may be cells implanted into the intervertebral disc prosthesis before, during, or after implantation into the patient. The cells that can invade an implanted intervertebral disc prosthesis of the present invention include, for example, vascular or neural cells, or chondrocytes. Such cell growth and penetration into an implanted intervertebral disc prosthesis can strengthen the prosthesis and resist slippage of the implant from the intervertebral space.

The present invention also provides various methods of using an intervertebral disc prosthesis to maintain an intervertebral space. In one aspect, the method of the present invention generally comprises a surgical procedure for anteriorly or posteriorly installing at least one intervertebral disc prosthesis into an intervertebral space between adjacent vertebrae while removing only a portion of the bony process of the adjacent vertebrae. The intervertebral disc prosthesis is slid between the adjacent vertebrae, thereby minimizing bone removal, reducing the risk of injury to the neural tissue, and minimizing trauma to the patient from the surgical procedure. It will be understood by those skilled in the art that the amount of bone removal required for placement of the intervertebral disc prosthesis within an intervertebral space will depend upon the conformation of the vertebrae and spinal column of the individual patient.

Thus, in this aspect, the method of the present invention comprises excising a portion of the intervertebral disc 21 separating adjacent vertebrae 20 to create a receiving slot. The method may further include removing material from at least one vertebra 20. At least one intervertebral disc prosthesis 10 is inserted into the intervertebral space 23 to support and maintain the separation of adjacent vertebrae 20. In another aspect, the method of the present invention may further include implanting a substantially rigid intervertebral spacer between adjacent vertebrae 20.

In yet another aspect, the method of the present invention also may include delivering a substance that, in the intervertebral space, has a consistency ranging from a semi-solid state to a solid state. Such semi-solid or solid materials may be used alone or to augment a full or partial intervertebral disc prosthesis or an intervertebral disc spacer. The augmenting substance may be in intimate contact with the intervertebral disc prosthesis or spacer, may be in proximate contact, or any combination thereof. By combining such materials, the desired vertebral flexibility and mobility for a particular application can be attained.

According to another aspect of the present invention described previously in commonly owned U.S. patent application Ser. No. 10/047,587, filed Jan. 15, 2002, and U.S. patent application Ser. No. 11/176,614, filed Jul. 7, 2005, both of which are incorporated by reference in their entirety, a biocompatible material may be used to fill all or a portion of an interior of an intervertebral disc. The disc may be at least partially hollowed to facilitate injection, if needed, or may be at least partially hollowed as a result of injury or herniation. The material inserted into the disc acts as a shock absorber to support the adjacent vertebrae.

If needed, a minimal portion of the interior disc tissue is removed prior to insertion of the filler material. Then, a filler material is injected, incorporated, implanted, poured, pumped, forced, or otherwise inserted (sometimes collectively “injected” or “inserted”) into the interior of the disc through an incision. The filler material generally is selected to provide the needed shock absorbency and flexibility to protect the spinal cord.

Thus, a prosthesis according to this aspect of the invention may be referred to as an “interior disc prosthesis”, since little or no exterior disc tissue is required to be removed prior to injection of the filler material. Thus, the intervertebral disc is able to be repaired without the need to remove any significant portion of the exterior of the disc. Instead, only an incision is needed to insert the material into the interior of the disc. The structure of the disc is maintained substantially.

The filler material may include any suitable solid, liquid, semi-solid, semi-liquid material, or any combination thereof capable of being injected into the interior of an intervertebral disc (sometimes collectively referred to as “injectable” materials). The filler material may be, for example, a multicomponent system or mixture of a solid and liquid, for example in a colloidal system (or “colloid”), a dispersion, a suspension, a gel, a sol, an emulsion, a latex, or any combination thereof. By way of example, and not by limitation, the filler material may include an encapsulated solid or liquid, or a plurality of particles, flakes, or fibers suspended, dispersed, or otherwise mixed in a suitable injectable medium or substance.

The filler material may be a thermoplastic, elastomeric, pseudoplastic, or dilatant material. It may be a Newtonian or non-Newtonian material. It may be an elastic, viscous, or viscoelastic material. It may be a thixotropic or rheopectic material. Any combination of properties is contemplated hereby for each of the components in the filler material and for the filler material.

The filler material may have a viscosity at 25° C. of from about 1 to about 150,000 centistokes (cSt). In one aspect, the filler material has a viscosity of from about 1 to about 20 cSt. In another aspect, the filler material has a viscosity of from about 20 to about 100 cSt. In yet another aspect, the filler material has a viscosity of from about 100 to about 500 cSt. In still another aspect, the filler material has a viscosity of from about 500 to about 1000 cSt. In another aspect, the filler material has a viscosity of from about 1000 to about 2000 cSt. In yet another aspect, the filler material has a viscosity of from about 2000 to about 5000 cSt. In a further aspect, the filler material has a viscosity of from about 5000 to about 10,000 cSt. In yet another aspect, the filler material has a viscosity of from about 1 to about 10,000 cSt. In another aspect, the filler material has a viscosity of from about 10,000 to about 50,000 cSt. In yet another aspect, the filler material has a viscosity of from about 50,000 to about 100,000 cSt. In still another aspect, the filler material has a viscosity of from about 100,000 to about 250,000 cSt.

Examples of solid components that may be suitable for use with the present invention include, for example, polymers, inorganic materials, or human or animal tissue. A polymeric component used in accordance with this aspect of the invention may be, for example, a thermoplastic, elastomeric, or pseudoplastic material. The liquid component typically is water.

In one example, the filler material is a hydrogel. As used herein, the term “hydrogel” refers to a multicomponent system including a three-dimensional network of polymer chains and water that fills the space between macromolecules. The term “hydrogel” includes pseudogels and chemical gels. Examples of materials that may be suitable for forming a hydrogel in accordance with the present invention include, but are not limited to, poly(ethylene oxide), poly(vinyl alcohol), polyvinylpyrrolidone and poly(hydroxyethyl methacrylate), glyceryl monooleate, glyceryl monostearate, glyceryl monooleate hydrolyzed gelatin, various polysaccharides, gelatin crosslinked with polyethylene glycol, and any combination thereof.

The present invention also contemplates use of a multi-part or multi-component system in which a substance or combination of substances forms a filler material, for example, a gel, in situ. Such materials may be a readily injectible liquid or particulate that forms a filler material upon contact with another substance. Such materials may increase in viscosity upon 5 combination, or may decrease, as needed or desired for a particular application. The substance or substances may be combined and injected into the interior of the disc prior to forming the gel, or may be injected as separate components that form a filler material when inside of the disc.

As shown in FIG. 4A and FIG. 4B, one example of an interior disc prosthesis 10 according the present invention is illustrated. A ribbon 16 or strip of resilient biocompatible 10 filler material may be removed from a sheet 14 of the biocompatible material. The sheet 14 of resilient biocompatible material may include at least one predetermined line 17 selected from an indentation, a plurality of indentations, or a plurality of partial perforations that facilitate the removal of the ribbon 16 from the sheet 14. The amount of the material to be implanted into an intervertebral disc may be selected readily, thereby accommodating variations in the size of the intervertebral disc 21 in different patients or between different pairs of vertebrae 20.

As stated above, the ribbon 16 or other injectable filler material may be inserted into the interior of an intervertebral disc 21 that has been at least partially hollowed by surgical removal of intervertebral disc tissue or as a result of disc herniation. Advantageously, the injectable filler material may be inserted into the disc with a minimal removal of tissue, if any, to access the implantation site. Once the site is accessed, the ribbon 16 may be inserted through a narrow incision 18 into the intervertebral disc 21. The intervertebral disc remains substantially intact.

With respect to the above description, it is to be realized that the optimum dimensional relationships for the parts of the invention, to include variations in size, materials, shape, form, function and manner of operation, assembly, and use, are deemed readily apparent and obvious to one skilled in the art, and all equivalent relationships to those illustrated in the drawing and described in the specification are intended to be encompassed by the present invention. Further, the various components of the embodiments of the invention may be interchanged to produce further embodiments and these further embodiments are intended to be encompassed by the present invention.

Although the invention has been described in detail for the purpose of illustration, it is understood that such detail is solely for that purpose, and variations can be made therein by those skilled in the art without departing from the spirit and scope of the invention which is defined by the following claims.

Accordingly, it will be readily understood by those persons skilled in the art that, in view of the above detailed description of the invention, the present invention is susceptible of broad utility and application. Many adaptations of the present invention other than those herein described, as well as many variations, modifications, and equivalent arrangements will be apparent from or reasonably suggested by the present invention and the above detailed description thereof, without departing from the substance or scope of the present invention.

Although numerous embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are used only for identification purposes to aid the reader's understanding of the embodiments of the present invention, and do not create limitations, particularly as to the position, orientation, or use of the invention unless specifically set forth in the claims. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It will be recognized by those skilled in the art, that various elements discussed with reference to the various embodiments may be interchanged to create entirely new embodiments coming within the scope of the present invention. It is intended that all matter contained in the above description or shown in the accompanying drawings shall be interpreted as illustrative only and not limiting. Changes in detail or structure may be made without departing from the spirit of the invention as defined in the appended claims. The detailed description set forth herein is not intended nor is to be construed to limit the present invention or otherwise to exclude any such other embodiments, adaptations, variations, modifications, and equivalent arrangements of the present invention.

What is claimed is:

1. A filler material for an intervertebral disc comprising a biocompatible material capable of being injected into an interior of an intervertebral disc through an incision in an exterior of the intervertebral disc.

2. The filler material of claim 1, comprising a hydrogel.

3. The filler material of claim 2, wherein the hydrogel comprises poly(ethylene oxide), poly(vinyl alcohol), polyvinylpyrrolidone and poly(hydroxyethyl methacrylate), glyceryl monooleate, glyceryl monostearate, glyceryl monooleate hydrolyzed gelatin, various polysaccharides, gelatin crosslinked with polyethylene glycol, or any combination thereof.

4. A method of maintaining an intervertebral space between adjacent vertebrae, comprising:
(a) making an incision in an intervertebral disc, the disc having an interior; and
(b) injecting a biocompatible material through the incision into the interior of the intervertebral disc,
wherein the biocompatible material comprises a colloid, a dispersion, a suspension, a gel, a sol, an emulsion, a latex, or any combination thereof.

5. The method of claim 4, wherein the biocompatible material comprises poly(ethylene oxide), poly(vinyl alcohol), polyvinylpyrrolidone and poly(hydroxyethyl methacrylate), glyceryl monooleate, glyceryl monostearate, glyceryl monooleate hydrolyzed gelatin, various polysaccharides, gelatin crosslinked with polyethylene glycol, or any combination thereof.

6. A method of maintaining an intervertebral space between adjacent vertebrae, comprising:
(a) making an incision in an intervertebral disc, the disc having an exterior and an interior; and
(b) inserting through the incision a biocompatible, compressible, resilient ribbon.

7. The method of claim 6, further comprising excising a portion of the interior of the intervertebral disc.

8. The method of claim 6, wherein the biocompatible, compressible, resilient ribbon is removed from a sheet of biocompatible, compressible, resilient material.

9. A method of maintaining an intervertebral space between adjacent vertebrae, comprising:
(a) making an incision in an intervertebral disc, the disc having an exterior and an interior; and
(b) injecting through the incision a plurality of components capable of forming a filler material when combined, the components maintained separately until immediately prior to injection into the interior of the intervertebral disc.
